(19) United States
(12) Patent Application Publication
(10) Pub. No.: US 2005/0282912 A1
(43) Pub. Date: Dec. 22, 2005

(54) Abnormal cannabidiols as neuroprotective agents for the eye

(76) Inventors: June Chen, San Juan Capistrano, CA (US); David F. Woodward, Lake Forest, CA (US)

Correspondence Address: Robert J. Baran (T2-7H);ALLERGAN, INC., Legal Department, 2525 Dupont Drive, Irvine, CA, 92612, US

(21) Appl. No.: 10/874,440

(22) Filed: Jun. 22, 2004

(51) Int. Cl. A61K 31/05

(52) U.S. Cl. 514/734

(57) ABSTRACT

The invention relates to the use of Abnormal Cannabidiols as neuroprotective agents. In particular said compounds are represented by the formula I

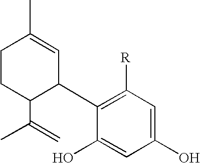

wherein R is selected from the group consisting of (CH₂)ₓ wherein x is 0 or an integer of from 1 to 7.

### FIELD OF THE INVENTION

The present invention relates to the use of Abnormal Cannabidiols to provide a neuroprotective effect to the eye of a mammal.

### BACKGROUND OF THE INVENTION

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupillary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical β-adrenoreceptor antagonists have traditionally been the drugs of choice for treating glaucoma.

It has long been know that one of the sequelae of glaucoma is damage to the optic nerve head. This damage, referred to as “cupping”, results in depressions in areas of the nerve fiber of the optic disk. Loss of sight from this cupping is progressive and can lead to blindness if the condition is not treated effectively.

Unfortunately lowering intraocular pressure by administration of drugs or by surgery to facilitate outflow of the aqueous humor is not always effective in obviating damage to the nerves in glaucomatous conditions. This apparent contradiction is addressed by Cioffi and Van Buskirk [Surv. of Ophthalmol., 38, Suppl. p. S107-16, discussion S116-17, May 1994] in the article, “Microvasculature of the Anterior Optic Nerve”. The abstract states:
The traditional definition of glaucoma as a disorder of increased intraocular pressure (IOP) oversimplifies the clinical situation. Some glaucoma patients never have higher than normal IOP and others continue to develop optic nerve damage despite maximal lowering of IOP. Another possible factor in the etiology of glaucoma may be regulation of the regional microvasculature of the anterior optic nerve. One reason to believe that microvascular factors are important is that many microvascular diseases are associated with glaucomatous optic neuropathy.

Subsequent to Cioffi, et al., Matusi published a paper on the “Ophthalmologic aspects of Systemic Vasculitis” [Nippon Rinsho, 52 (8), p. 2158-63, August 1994] and added further support to the assertion that many microvascular diseases are associated with glaucomatous optic neuropathy. The summary states:
Ocular findings of systemic vasculitis, such as polyarteritis nodosa, giant cell angitis and aortitis syndrome were reviewed. Systemic lupus erythematosus is not categorized as systemic vasculitis, however its ocular findings are microangiopathic. Therefore, review of its ocular findings was included in this paper. The most common fundus finding in these diseases is ischemic optic neuropathy or retinal vascular occlusions. Therefore several points in diagnosis or pathogenesis of optic neuropathy and retinal and choroidal vaso-occlusion were discussed. Choroidal ischemia has come to be able to be diagnosed clinically, since fluorescein angiography was applied in these lesions. When choroidal arteries are occluded, overlying retinal pigment epithelium is damaged. This causes disruption of barrier function of the epithelium and allows fluid from choroidal vasculatures to pass into subsensory retinal spaces. This is a pathogenesis of serous detachment of the retina. The retinal arterial occlusion formed non-perfused retina. Such hypoxic retina released angiogenesis factors which stimulate retinal and iris neovascularizations and iris neovascularizations may cause neovascular glaucoma.

B. Schwartz, in “Circulatory Defects of the Optic Disk and Retina in Ocular Hypertension and High Pressure Open-Angle Glaucoma” [Surv. Ophthalmol., 38, Suppl. pp. S23-24, May 1994] discusses the measurement of progressive defects in the optic nerve and retina associated with the progression of glaucoma. He states:
Fluorescein defects are significantly correlated with visual field loss and retinal nerve fiber layer loss. The second circulatory defect is a decrease of flow of fluorescein in the retinal vessels, especially the retinal veins, so that the greater the age, diastolic blood pressure, ocular pressure and visual field loss, the less the flow. Both the optic disk and retinal circulation defects occur in untreated ocular hypertensive eyes. These observations indicate that circulatory defects in the optic disk and retina occur in ocular hypertension and open-angle glaucoma and increase with the progression of the disease.

Thus, it is evident that there is an unmet need for agents that have neuroprotective effects in the eye that can stop or retard the progressive damage that occurs to the nerves as a result of glaucoma or other ocular afflictions.

Certain Abnormal Cannabidiols are disclosed in Howlett et al, “International Union of Pharmacology. XXVII. Classification of Cannabinoid Receptors”, Pharmacological Reviews 54: 161-202, 2002.

### SUMMARY OF THE INVENTION

We have found that Abnormal Cannabidiols are potent neuroprotective agents. We have further found that Abnormal Cannabidiols and homologues and derivatives thereof are especially useful in providing a neuroprotective effect to the eye of a mammal, e.g. a human.

The present invention relates to methods of providing a neuroprotective effect to the eye of a mammal, e.g. a human, which comprises administering an effective amount of a compound represented by the formula I

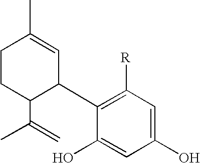

wherein R is selected from the group consisting of (CH₂)ₓ, wherein x is 0 or an integer of from 1 to 7.

In a further aspect, the present invention relates to pharmaceutical compositions comprising a therapeutically effective amount of a compound of formulae (I), in admixture with an non-toxic, pharmaceutically acceptable liquid vehicle.

### DETAILED DESCRIPTION OF THE INVENTION

The present invention relates to the use of Abnormal Cannabidiols and homologues and derivatives thereof as neuroprotective agents. These therapeutic agents are represented by compounds having the formula I

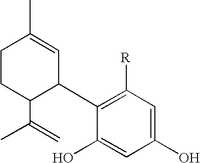

as defined above. The preferred compounds used in accordance with the present invention are encompassed by the following structural formula II 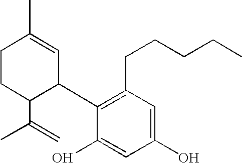
or formula III

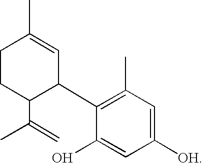

In all of the above formulae, as well as in those provided hereinafter, the straight lines represent bonds. Where there is no symbol for the atoms between the bonds, the appropriate carbon-containing radical is to be inferred. For example in formula II, the radical extending from the phenyl ring is a polymethylene (CH₂) radical terminated with a methyl radical, i.e. a butylenylmethyl radical.

Pharmaceutical compositions may be prepared by combining a therapeutically effective amount of at least one compound according to the present invention, or a pharmaceutically acceptable salt thereof, as an active ingredient, with conventional ophthalmically acceptable pharmaceutical excipients, and by preparation of unit dosage forms suitable for topical ocular use. The therapeutically efficient amount typically is between about 0.0001 and about 5% (w/v), preferably about 0.001 to about 1.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional, pharmaceutically acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 0-10 |
| buffer | 0.01-10 |
| pH adjustor | q.s. pH 4.5-8.0 |
| antioxidant | as needed |
| surfactant | as needed |
| purified water | as needed to make |
| | 100% |

The actual dose of the active compounds of the present invention depends on the specific compound, and on the condition to be treated; the selection of the appropriate dose is well within the knowledge of the skilled artisan.

The ophthalmic formulations for use in the method of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses.

Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

The invention is further illustrated by the following non-limiting Examples.

### EXAMPLE 1

Abnormal Cannabidiol, also named as Abn-CBD (4-[(1R,6R)-3-Methyl-6-(1-methylenthenyl)-2-cyclohexen-1-yl]-5-pentyl-1,3-benzenediol, M.W. 314.47, may be purchased from Tocris Cookson Inc., Ellisville, Mo., USA.

The above compound is well known and may be purchased or synthesized by methods known in the art.

### EXAMPLE 2

### Method of Measuring a Neuroprotective Effect

The dissection and dissociation of the rat hippocampal neuron cell cultures is carried out. Briefly, whole cerebral neocortices are removed from fetal rats, gestation age 15-19 days and kept in calcium free and magnesium free Hanks' balanced salt solution. The hippocampi are removed under a dissecting microscope and the meninges are stripped away. When all the hippocampi are removed, the tissues are incubated in 0.05% trypsin solution for 30 minutes at 37° C. At the end of 340 minutes, the trypsin solution is replaced with plating medium (minimal essential medium supplemented with 2% Hyclone horse serum, 1% fetal calf serum, 25 mM glucose, 1% glutamine and 1% penicillin/streptomycin and N₂ supplement). Then the tissues are triturated with a Pasteur pipette 10 times and then again with a pipette whose tip has been fire polished to about half the normal diameter. The dissociated neuronal cells then are plated on poly D-lysine coated, 15 mm 24 well plates (2×10⁵ cells/well) in plating medium.

The cell cultures are kept at 37° C. in a humidified, 5% CO₂ containing atmosphere. After 1-2 days, the horse serum level in the plating media is increased to 8%. After 4-7 days, the non-neuronal cell division is halted by 24 hours exposure to 10⁻⁶M Cytosine arabinoside (ARA-C), and the cells are then placed into growing medium with 4% horse serum, 1% fetal calf serum, 25 mM glucose, 1% glutamine and 1% penicillin/streptomycin and N₂ supplement. Subsequent medium replacement is carried out every other day until the neuronal cells mature (15-20 days). Only matured cell cultures are selected for study.

Exposure of the excitatory amino acids is performed in minimal essential medium (MEM). Extreme care is taken to wash out the growing medium from cultures before the addition of the excitatory amino acid since the neurons are very sensitive to disturbance. Matured cell cultures are exposed to either glutamate, α-amino-3-hydroxy-5-methyl-4-isoxazole propionic acid (AMPA), N-methyl-D-aspartate (NMDA), or kainic acid.

Cytotoxicity or cell injury is scored by light microscopy examination with trypan blue. In most experiments, the overall neuronal cell injury is quantitated by the amount of lactate dehydrogenase (LDH) released by the damaged cells into the media 24 hours after drug exposure.

LDH is measured at room temperature using Promega non-radioactive cytotoxicity assay kit. The absorbance of the reaction mixture is measured at 490 nm.

The effect of the Abnormal Cannabidiol of Example 1 on NMDA-induced neurotoxicity shows that the compound of Example 1 has a neuroprotective effect.

### EXAMPLE 3

### Determination of Abnormal Cannabidiol Activity

Abnormal Cannabidiol receptor activity may be measured in accordance with the procedure disclosed in (Wagner J A et al., Hypertension 33 [part II], 429 (1999); Járai Z et al., PNAS 96, 14136 (1999), which is hereby incorporated by reference in its entirety.

### EXAMPLE 4

### Method of Measuring a Neuroprotective Effect

The Experiment of Example 2 is repeated with other Abnormal Cannabidiols and the results are essentially as shown for the compound of Example 1.

The foregoing description details specific methods and compositions that can be employed to practice the present invention, and represents the best mode contemplated. However, it is apparent from one of ordinary skill in the art that different pharmaceutical compositions may be prepared and used with substantially the same results. That is, other Abnormal Cannabidiols, will effectively provide neuroprotection in animals and are within the broad scope of the present invention.

1. A method of providing a neuroprotective effect to the eye of a mammal which comprises applying to the eye an amount sufficient to treat ocular hypertension of a compound of formula I 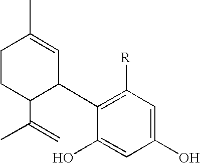
wherein R is selected from the group consisting of (CH₂)ₓ wherein x is 0 or an integer of from 1 to 7.

2. The method of claim 1 wherein said compound is a compound of the formula II 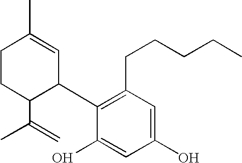
or formula III

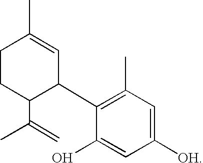

3. An ophthalmic solution having a neuroprotective effect comprising a therapeutically effective amount of a compound of formula I 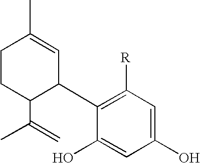
wherein R is selected from the group consisting of (CH₂)ₓ, wherein X is 0 or an integer of from 1 to 7.

4. A method for providing a neuroprotective effect to the eye of a mammal which comprises applying to the eye an amount sufficient to provide ocular neuroprotection of a compound having Abnormal Cannabidiol activity.

5. A method of protecting the retinal or optic nerve cells in a mammal suffering a noxious action or at risk of experiencing a noxious action on said nerve cells comprising administering to said mammal an effective amount of a compound of formula I to inhibit or prevent nerve cell injury or death 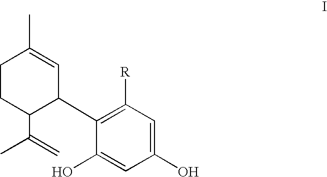
wherein R is selected from the group consisting of (CH₂)ₓ wherein x is 0 or an integer of from 1 to 7.

6. The method of claim 5 wherein the noxious action is the elevated intraocular pressure of glaucoma.

7. The method of claim 5 wherein the noxious action is ischemia associated with glaucoma.

8. The method of claim 5 wherein the noxious action is diabetic retinopathy.

9. The method of claim 5 wherein the noxious action is non-glaucomatous ischemia.

10. The method of claim 5 wherein the noxious action is microangiopathic in nature and is a symptom of the disease chosen from the group consisting of polyarteritis nodosa, giant cell angitis, aortitis syndrome and systemic lupus erythematosus.

11. The method of claim 5 wherein oral administration is used to supply the compound to the mammal systemically.

12. The method of claim 5 wherein intrabulbar injection in the eye is used to supply the compound to the mammal.

13. The method of claim 5 wherein parenteral administration is used to 5 supply the compound to the mammal systemically.

14. The method of claim 5 wherein intramuscular injection is used to supply the compound to the mammal systemically.
